(19) United States
(12) Patent Application Publication
(10) Pub. No.: US 2023/0142920 A1
(43) Pub. Date: May 11, 2023

(54) KITS AND METHODS FOR DETECTING MARKERS

(71) Applicant: EDP Biotech Corporation, Knoxville, TN (US)

(72) Inventors: Herbert A. Fritsche, Houston, TX (US); Jason L. Liggett, Knoxville, TN (US)

(21) Appl. No.: 18/152,008

(22) Filed: Jan. 9, 2023

Related U.S. Application Data
(62) Division of application No. 16/502,599, filed on Jul. 3, 2019.
(60) Provisional application No. 62/694,390, filed on Jul. 5, 2018.

(51) Int. Cl. G01N 33/574; G16H 50/30; G16B 40/10; G01N 33/541; G01N 33/543

(52) U.S. Cl. CPC G01N 33/57419; G16H 50/30; G16B 40/10; G01N 33/541; G01N 33/54326

(57) ABSTRACT

This disclosure provides kits and methods for detecting markers in a sample from a subject with unknown status and generating a risk assessment of the presence or absence of cancer, such as colorectal cancer. In embodiments, a kit comprises at least two reagents, each specifically binding to one of at least two polypeptides in a sample from the subject. The polypeptides include IL-8 and ferritin. The kit further includes at least one standard comprising a known amount of at least one of the polypeptides. The kit can also include computer readable media comprising instructions to analyze the detected amounts of the at least two polypeptides using a machine learning algorithm to determine whether a subject has an increased risk of the presence of colorectal cancer.

### CROSS REFERENCE TO RELATED APPLICATION

This application is a divisional of U.S. Ser. No. 16/502,599, titled KITS AND METHODS FOR DETECTING MARKERS, filed on Jul. 3, 2019, which claims priority to U.S. Ser. No. 62/694,390, titled KITS AND METHODS FOR DETECTING MARKERS, filed on Jul. 5, 2018, the disclosure of which is hereby incorporated by reference in its entirety.

### TECHNICAL FIELD

This disclosure provides kits and methods for detecting markers in a sample from a subject and determining the presence of or risk of the presence of cancer, such as colorectal cancer.

### BACKGROUND

Colorectal cancer (CRC) is the second leading cause of cancer-related deaths in the U.S. The survival rate for patients diagnosed with CRC is highly dependent on when it is caught. CRC usually progresses through stages from adenomatous polyps to Stage I, Stage II, Stage III, and Stage IV. Adenomatous polyps can be classified as low risk or high risk polyps depending on size, number, high grade dysplasia, and villous features. Stages I and II are local stages, during which aberrant cell growth is confined to the colon or rectum. Stage III is a regional stage, meaning the cancer has spread to the surrounding tissue but remains local. Stage IV is distal and indicates that the cancer has spread throughout the other organs of the body, most commonly the liver or lungs. It is estimated that the five-year survival rate is over 90% for those patients diagnosed with Stage I CRC, compared to 13% for a Stage IV diagnosis. Colorectal cancer is one of the more preventable and treatable cancers given its typically slow progression from early stages to metastatic disease but it is one of the least prevented cancers. This is at least partly due to the poor compliance with available screening by patients due to the invasive or unpleasant nature of the current screening tests.

The current screening assays in widespread use for the diagnosis of colorectal cancer are the fecal occult blood test (FOBT), fetal immunochemical test (FIT), flexible sigmoidoscopy, and colonoscopy. FOBT has relatively low specificity resulting in a high rate of false positives. All positive FOBT must therefore be followed up with colonoscopy. Sampling is done by individuals at home and requires at least two consecutive fecal samples to be analyzed to achieve sufficient sensitivity. Some versions of the FOBT also require dietary restrictions prior to sampling. FOBT also lacks sensitivity for early stage cancerous lesions that do not bleed into the bowel. These are the lesions for which treatment is most successful.

Numerous serum markers, such as carcinoembryonic antigen (“CEA”), carbohydrate antigen 19-9, and lipid-associated sialic acid, have been investigated in colorectal cancer. However, their low sensitivity has led to recommendation that these markers are not suitable for screening tests. Thus there remains a need to provide kits and methods for detecting markers for colorectal cancer in an assay with higher levels of sensitivity.

### SUMMARY

The methods and kits as described include the detection of one or more markers in a sample from a subject of unknown status. In embodiments, the detection of a combination of markers is useful to assess the presence of or the risk of the presence of cancer, to determine if further examination of the colon for cancer should be conducted, and/or for administering or monitoring treatment. In embodiments, the sample comprises blood, plasma, serum, saliva, sweat, urine, or feces. In embodiments, the sample comprises circulating tumor cells, exosomes, and/or methylated DNA. The sample can be obtained as a part of a routine screening during a checkup, upon suspicion of the presence of cancer, during treatment, upon completion of treatment, and/or as a periodic follow-up following remission of the cancer. In embodiments, the cancer is colorectal cancer.

In embodiments, a method for detecting at least four different markers in a sample from a subject with unknown status comprises detecting at least four different polypeptides and/or nucleic acids coding for the polypeptides in the sample by contacting the sample with at least four different reagents, each reagent specifically detecting the presence and/or an amount of one of the at least four different polypeptides and/or nucleic acids coding for the polypeptides, wherein the at least four polypeptides comprise GDF15, keratin 1-10, and two or more of hepsin, IL-8, and CEA, L1CAM, MCP-1, and OPG; and determining whether the combination of the presence and/or detected amounts of each of the at least four different polypeptides and/or nucleic acids coding for the polypeptides is indicative of the presence of or an increased risk of the presence of colorectal cancer. In embodiments, the presence of or the increased risk of the presence of colorectal cancer can be stratified into low risk adenomatous polyps, high risk adenomatous polyps, Stage I, Stage II, Stage III, or Stage IV.

In embodiments, a blood sample is obtained from the subject and the amounts of at least four different markers are detected. In embodiments, the sample is a serum sample, a blood sample, a plasma sample, a urine sample, a tissue sample, a feces sample, or a saliva sample. In embodiments, the sample comprises circulating tumor cells, exosomes, tumor nucleic acids, methylated DNA, and combinations thereof.

In embodiments, one or more additional markers are detected including, without limitation, AFP, ferritin, CATD, CD44, ALDH1A1, EPCAM, FAP, Galectin 3, IL-6, kallikrein 6, CEA, keratin 6, L1CAM, MIA, midkine (MDK), TWEAK, NSE, ON (SPARC), TGM2, VEGFA, YKL40, and combinations thereof. In embodiments, additional markers are detected including MCP-1 and OPG.

In embodiments, the plurality of polypeptides comprise GDF15, kertain 1-10, hepsin, and IL-8. In embodiments, the plurality of polypeptides comprise GDF15, kertain 1-10, CEA, L1CAM, MCP-1, and OPG. In embodiments, the plurality of polypeptides comprise GDF15, kertain 1-10, CEA, L1CAM, hepsin, IL-8, MCP-1, and OPG.

In embodiments, the at least four different reagents comprise one or more primary antibodies or antigen binding fragments thereof, each primary antibody or antigen binding fragment thereof specifically binds to one of the plurality of polypeptides comprising GDF15, keratin 1-10, and two or more of hepsin, IL-8, CEA, L1CAM, MCP-1, and OPG. In embodiments, the at least four different primary antibodies or antigen binding fragments thereof are attached to a solid surface. In some embodiments, each of the at least four different primary antibodies or antigen binding fragments thereof are attached to a different solid surface. In some embodiments, each of the different solid surfaces has a different internal marker. In embodiments, each of the different solid surfaces is the same type of solid surface but differs only in the type of internal marker. In some embodiments, a solid surface comprises a bead, a magnetic bead, a well, slide, or a tube. In some embodiments, the internal marker comprises a fluorescent dye, a quantum dot, a protein tag, a RFID tag, or combinations thereof.

In embodiments, each of the at least four different reagents can each be in a separate container or location on a solid surface. In other embodiments, the at least four different reagents can be in a single container or single location on a solid surface. In yet other embodiments, at least two of the at least four different reagents can be in a single container or single location on a solid surface.

In embodiments, a method further comprises contacting the sample with at least four detectably labelled secondary reagents, each detectably labelled secondary reagent specifically detects or binds to one of the at least four polypeptides; and each of the at least four detectably labelled secondary reagents has a different detectable label. In embodiments, the detectable label comprises a fluorescent dye, a radiolabel, a protein or peptide tag, an enzyme, or a luminescent reactant. In embodiments, the label on the secondary reagent is different than the internal label on the solid surface. In some embodiments, the at least four detectably labelled secondary reagents comprise a secondary antibody or antigen binding fragments thereof; each secondary antibody or antigen binding fragment thereof specifically binds to one of the at least four polypeptides. In embodiments, the secondary antibody or antigen binding fragment thereof binds to a different epitope than the primary antibody specific for the same polypeptide.

In embodiments, a method further comprises contacting the at least four different reagents with a standard comprising a known amount of at least one of the four different polypeptides and determining the amount of the at least one polypeptide in the standard. In embodiments, a standard comprises all of the at least four polypeptides.

In embodiments, a standard can be a low concentration quality control sample, and/or a high concentration quality control standard. In some embodiments, the concentration of the polypeptide in the low quality control standard ranges from 0.001 to 500 ng/ml depending on the polypeptide. For example, a low concentration quality control standard for GDF15 is about 0.01 to about 0.5 ng/ml, a low concentration quality control standard for hepsin is about 2 to about 10 ng/ml, a low concentration quality control standard for IL-8 is about 0.1 to about 0.5 ng/ml, and a low concentration quality control standard for keratin 1-10 is about 100 to about 500 ng/ml.

In some embodiments, the concentration of the polypeptide in the high quality control standard ranges from 0.05 to 5000 ng/ml depending on the polypeptide. For example, a high concentration quality control standard for GDF15 is about 1 to about 5 ng/ml, a high concentration quality control standard for hepsin is about 20 to about 50 ng/ml, a high concentration quality control standard for IL-8 is about 0.1 to about 1 ng/ml, and a high concentration quality control standard for keratin 1-10 is about 1000 to about 5000 ng/ml.

In embodiments, a method further comprises determining the accuracy of the measurement of the detected amounts of each of the polypeptides by determining the percent coefficient of variation for each of the polypeptides based on the detected amount of the standard for each of the polypeptides.

In embodiments, determining if the combination of the detected amounts of the at least four polypeptides in the sample is indicative of the presence of or an increased risk of the presence of colorectal cancer is determined using a supervised machine learning algorithm. In embodiments, Model 1 uses the Universal Process Classification algorithm, a variant of K-Nearest Neighbors classification that utilizes a distance measurement to identify nearest neighbors. In embodiments, Model 2 uses support vector classifiers with radial basis function kernels during identification. In embodiments, Model 3 is a random forest classifier, an algorithm class that makes predictions by averaging the results of multiple randomly-initialized binary decision trees.

In certain embodiments, determining if the combination of the detected amounts of the at least four polypeptides in the sample is indicative of the presence of or an increased risk of the presence of colorectal cancer comprises: receiving the detected amount of each of the polypeptides on a computing device; retrieving a coefficient for each of the detected amounts of each of the polypeptides from a database on the computing device; multiplying each of the detected amounts by the corresponding coefficient to generate a weighted level for each of the polypeptides on the computing device; analyzing the combination of weighted levels for each polypeptide with a model on the computing device to determine if the subject has colorectal cancer or an increased risk of the presence of colorectal cancer based on a change or lack thereof in the combination of weighted levels for each of the polypeptides detected in the sample from the subject with unknown status to the combination of predetermined weighted values of the polypeptides for normal subjects. In embodiments, a method further comprises generating an output on the computing device indicating the presence of or the risk of the presence of colorectal cancer in the subject.

In embodiments, in the methods described herein, the output provides the current status of the subject or a risk assessment of the current status of the subject. In embodiments, the current status is colorectal cancer present or not present. In other embodiments, the output provides stratification of the presence of or risk of the presence of low risk adenomatous polyps, high risk adenomatous polyps, stage I, stage II, stage III, or stage IV colorectal cancer.

In embodiments, if the sample from the subject indicates the presence of or the risk of the presence of colorectal cancer, the subject can undergo an examination of the colon for cancer such as by a colonoscopy, a sigmoidoscopy, a biopsy, a CAT scan, or MRI. In embodiments, if the sample from the subject indicates the presence of colorectal cancer or increased risk of colorectal cancer, whether or not identified by additional testing, the subject can be treated with a treatment for colorectal cancer. In embodiments, a treatment regimen can be selected depending on whether the sample for the subject indicates whether the subject has adenomatous polyps or stage I colorectal cancer versus Stage III or IV colorectal cancer. Subjects having Stage III or IV colorectal cancer may receive a more aggressive treatment regimen.

In embodiments, a kit comprises at least four different reagents; each reagent specifically binds to one of at least four different polypeptides and/or nucleic acids coding for the polypeptides in a sample from the subject,; and at least one standard comprising a known amount of at least one of the at least four different polypeptides and/or nucleic acids coding for the polypeptides,. In embodiments, each of the at least four different reagents is a primary antibody or antigen binding fragment thereof that specifically binds to one of the at least four different polypeptides.

In embodiments, a kit comprises a computer readable medium containing instructions for analyzing the combination of the detected amount of each of the polypeptides and/or nucleic acids coding for the polypeptides from a subject with unknown status with a mathematical model to generate a risk assessment of the current status of the subject as having or not having colorectal cancer. In embodiments, the mathematical model employed is a supervised machine learning algorithm. In embodiments, Model 1 uses the Universal Process Classification algorithm, a variant of K-Nearest Neighbors classification that utilizes a distance measurement to identify nearest neighbors. In embodiments, Model 2 uses support vector classifiers with radial basis function kernels during identification. In embodiments, Model 3 is a random forest classifier, an algorithm class that makes predictions by averaging the results of multiple randomly-initialized binary decision trees. In embodiments, Model 4 is also a random forest classifier.

In embodiments, the analysis of the combination of the detected amounts of each of the polypeptides and/or nucleic acids coding for the polypeptides is conducted using an internet accessible supervised machine learning algorithm.

In embodiments, one or more non-transitory computer-readable media have computer-executable instructions embodiment thereon that, when executed by one or more computing devices, cause the computing device to: receive the detected amount of each of the polypeptides coding for the polypeptides; retrieve a coefficient for each of the detected amounts of each of the polypeptides from a database; multiply each of the detected amount of the polypeptides by the corresponding coefficient to generate a weighted level for each of the polypeptides; analyze the combination of weighted levels for each polypeptide with a model to determine the probability that the subject has colorectal cancer or is normal based on a change or lack thereof from the combination of predetermined weighted values of the polypeptides for normal subjects.

In other embodiments, a kit comprises a computer readable medium containing instructions to access a database of profiles of the combination of the detected amount of each of the polypeptides and/or nucleic acids coding for the polypeptides from subjects having stage I colorectal cancer, stage II colorectal cancer, stage III colorectal cancer, stage IV colorectal cancer, high risk adenomatous polyps, low risk adenomatous polyps, and/or normal subjects; and to determine whether the profile from the subject with unknown status is similar to any of the profiles from subjects with known status to identify whether the subject with unknown status has stage I colorectal cancer, stage II colorectal cancer, stage III colorectal cancer, stage IV colorectal cancer, high risk adenomatous polyps, low risk adenomatous polyps, or is normal.

In embodiments, each of the at least four different reagents can each be in a separate container or separate location on a solid surface. In other embodiments, the at least four different reagents can be in a single container or single location on a solid surface. In yet other embodiments, at least two of the at least four different reagents can be in a single container or single location on a solid surface.

In embodiments, a kit further comprises at least four detectably labelled secondary reagents, each detectably labelled secondary reagent specifically binds to one of the at least four polypeptides; and each of the at least four detectably labelled secondary reagents has a different detectable label. In embodiments, the detectable label comprises a fluorescent dye, a radiolabel, a protein or peptide tag, an enzyme, or a luminescent reactant. In embodiments, the label on the secondary reagent is different than the internal label on the solid surface. In some embodiments, the at least four detectably labelled secondary reagents comprise a secondary antibody or antigen binding fragments thereof; each secondary antibody or antigen binding fragment thereof specifically binds to one of the polypeptides. In embodiments, the secondary antibody or antigen binding fragment thereof binds to a different epitope than the primary antibody for the same polypeptide.

In embodiments, the at least four reagents are attached to a solid surface. In some embodiments, the solid surface comprises a bead, a magnetic bead, a well, slide, a tube, or combinations thereof. In yet other embodiments, each of the at least four reagents is attached to a different solid surface; each of the different solid surfaces having a different internal marker. In embodiments, the different internal marker comprises a fluorescent dye, a quantum dot, a protein tag, a RFID tag, or combinations thereof. In embodiments, the internal marker of the solid surface is different than each of the detectable labels of the detectably labelled secondary reagents.

In embodiments, a kit further comprises a standard comprising a known amount of at least one of the four polypeptides. In embodiments, a standard comprises a known amount of each of the at least four polypeptides. In embodiments, a standard comprises all of the at least four polypeptides.

In embodiments, a standard can be a low concentration quality control sample, and/or a high concentration quality control standard. In some embodiments, the concentration of the polypeptide in the low quality control standard ranges from 0.001 to 500 ng/ml depending on the polypeptide. For example, a low concentration quality control standard for GDF15 is about 0.01 to about 0.5 ng/ml, a low concentration quality control standard for hepsin is about 2 to about 10 ng/ml, a low concentration quality control standard for IL-8 is about 0.1 to about 0.5 ng/ml, and a low concentration quality control standard for keratin 1-10 is about 100 to about 500 ng/ml.

In some embodiments, the concentration of the polypeptide in the high quality control standard ranges from 0.05 to 5000 ng/ml depending on the polypeptide. For example, a high concentration quality control standard for GDF15 is about 1 to about 5 ng/ml, a high concentration quality control standard for hepsin is about 20 to about 50 ng/ml, a high concentration quality control standard for IL-8 is about 0.1 to about 1 ng/ml, and a high concentration quality control standard for keratin 1-10 is about 1000 to about 5000 ng/ml.

In some embodiments, a kit further comprises a validation control. In embodiments, a validation control comprises a sample form a subject known to have low risk adenomatous polyps, high risk adenomatous polyps, stage I, stage II, stage III, or stage IV colorectal cancers. In embodiments, a validation control for each of low risk adenomatous polyps, high risk adenomatous polyps, stage I, stage II, stage III, or stage IV colorectal cancers is included in the kit.

### BRIEF DESCRIPTION OF THE DRAWINGS

FIG. 1 illustrates a schematic diagram of a kit for detecting polypeptides in a sample from a subject with unknown status.

FIG. 2 is a block diagram illustrating an example of the physical components of the computing device of FIG. 1.

FIG. 3 is a flow chart illustrating an example method of detecting at least four different polypeptides in a sample from a subject with unknown status using the kit of FIG. 1.

### DETAILED DESCRIPTION

### Definitions

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms “a,” “an”, and “the” include the plural reference unless the context clearly indicates otherwise. Thus, for example, the reference to an antibody is a reference to one or more such antibodies, including equivalents thereof known to those skilled in the art. Other than in the operating examples, or where otherwise indicated, all numbers expressing quantities of ingredients or reaction conditions used herein should be understood as modified in all instances by the term “about.” The term “about” when used in connection with numerical values means ±20% and with percentages means ±1%.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains.

For the purposes of this application the following terms shall have the following meanings:

As used herein, an “antigen” is a molecule or a portion of a molecule capable of being bound by an antibody. An antigen may have one or more than one epitope. An antigen will bind in a highly selective manner with its corresponding antibody and not with the multitude of other antibodies which may be evoked by other antigens.

As used herein, an “antibody” includes both intact immunoglobulin molecules as well as portions, fragments, peptides and derivatives thereof, such as, for example, Fab, Fab', F(ab')2, Fv, scFv, CDR regions, or any portion or peptide sequence of the antibody that is capable of binding antigen or epitope. An antibody is said to be “capable of binding” a molecule if it is capable of specifically reacting with the molecule to thereby bind the molecule to the antibody.

Antibody also includes chimeric antibodies, anti-idiotypic (anti-Id) antibodies to antibodies that can be labeled in soluble or bound form, as well as fragments, portions, regions, peptides or derivatives thereof, provided by any known technique, such as, but not limited to, enzymatic cleavage, peptide synthesis, phage display, or recombinant techniques. Antibody fragments or portions may lack the Fc fragment of intact antibody, clear more rapidly from the circulation, and may have less non-specific tissue binding than an intact antibody. Examples of antibody may be produced from intact antibodies using methods well known in the art, for example by proteolytic cleavage with enzymes such as papain (to produce Fab fragments) or pepsin (to produce F (ab′) 2 fragments). See e.g., Wahl et al., 24 J. Nucl. Med. 316-25 (1983). Portions of antibodies may be made by any of the above methods, or may be made by expressing a portion of the recombinant molecule. For example, the CDR region(s) of a recombinant antibody may be isolated and subcloned into the appropriate expression vector. See, e.g., U.S. Pat. No. 6,680,053.

As used herein, a “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F (ab′) 2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

As used herein, “alpha-1-antichymotrypsin”, or “ACT” refers to a polypeptide that has serine protease inhibitory activity. ACT is also known as SERPINA3, AACT, growth inhibiting protein 24 (GIG24), growth inhibiting protein 25 (GIG25), cell growth inhibiting gene 24/25 protein, and serine proteinase inhibitor clade A, member 3. A representative amino acid sequence of ACT is NP_001076/gI 50659080.

As used herein, “AFP” refers to alpha —fetoprotein, a plasma protein produced by the yolk sac and the liver during fetal development. A representative amino acid and nucleotide sequence for AFP is NP_001125, and NM_001134, respectively.

As used herein, “CATD” refers to cathepsin D, a pepsin like peptidase that plays a roles in protein turnover, and activation of hormones and growth factors. Cathepsin D is also known as CTSD. A representative amino acid and nucleotide sequence for CATD is NP_001900, and NM_001909, respectively.

As used herein, “CD44” refers to cluster differentiation antigen, a cell surface glycoprotein that is a receptor for hyaluronic acid and interacts with osteopontin, collagens, and matrix metalloproteinases. There are many functional distinct isoforms of this protein. In embodiments, the isoform includes amino acids 145-186 as shown in UniProt record P16070 for human CD44. A representative amino acid and nucleotide sequence for CD44 variant 6 is NP 001189484, and NM 001202555, respectively.

As used herein, “CEA” refers to carcinoembryonic antigen. CEA are glycosyl phosphatidyl inositol cell surface anchored proteins that serve as ligands for L-selectin and E-selectin. There are a number of different forms which are also identified as CD66 molecules. CEACAMS, without any glycosylation, has an exemplary amino acid sequence found in NP_004354;gI 98986445; Uniprot P06731-1.

As used herein, the term “colorectal cancer”, also known as “colon cancer”, “bowel cancer” or “rectal cancer”, refers to all forms of cancer originating from the epithelial cells lining the large intestine and/or rectum.

As used herein, “DKK-1” refers to dickkopf related protein 1, a secreted protein characterized by two cysteine rich domains that mediate protein-protein interactions. A representative amino acid sequence is found at NP_036374. A representative nucleotide sequence is found at NM_012242.

As used herein, “EPCAM” refers to epithelial cell adhesion molecule, a homotypic calcium independent adhesion molecule found on normal epithelial cells and gastrointestinal carcinomas. A representative amino acid and nucleotide sequence for EPCAM is NP_002345, and NM_002345, respectively.

As used herein, “FAP” refers to fibroblast activation protein, a homodimeric integral membrane gelatinase. This protein is also known as Seprase. A representative amino acid and nucleotide sequence for FAP is XP_011509098, and XM_011510796, respectively.

As used herein, “ferritin” refers to ferritin, an intracellular iron storage protein. A representative amino acid and nucleotide sequence for ferritin light chain is NP_000137, and NM_000146, respectively. A representative amino acid and nucleotide sequence for ferritin heavy chain is NP_002023, and NM_002032, respectively.

As used herein, “galectin-3” refer to a member of carbohydrate binding proteins, especially beta galactosidases. There are multiple isoforms of this protein. A representative amino acid and nucleotide sequence for galectin 3 is NP_001344607, and NM_001357678, respectively.

As used herein, “GDF15” refers to growth differentiation factor 15, secreted ligand of the TGF beta family of proteins and has cytokine activity. A representative amino acid and nucleotide sequence for GDF15 is NP_004855, and NM_004864, respectively.

As used herein, “hepsin” refers to a type two membrane serine protease. There are multiple isoforms of this protein. A representative amino acid and nucleotide sequence for hepsin is NP_002142, and NM_002151, respectively.

As used herein, “IL-8” refers to interleukin 8, a chemotactic and angiogenic factor. This protein is also known as CXC chemokine, CXCL8. A representative amino acid and nucleotide sequence for 11-8 is NP_000575, and NM_000584, respectively.

As used herein “keratin 6” refers to a type two cytokeratin found in epithelial tissues. There are multiple forms of keratin 6 including keratin 6A and keratin 6B. A representative amino acid and nucleotide sequence for keratin 6A is NP_005545, and NM_005554, respectively. A representative amino acid and nucleotide sequence for keratin 6B is NP_0055465 and NM_005554, respectively.

As used herein, “keratin 1-10” refers to a type two cytokeratin found in epithelial tissues and is expressed as a dimer with family member keratin 10, a type 1 acidic cytokeratin family. A representative amino acid and nucleotide sequence for keratin 1 is NP_006112, and NM_006121, respectively. A representative amino acid and nucleotide sequence for keratin 10 is NP_000412 and NM_000421, respectively.

As used herein, “MCP-1” refers to monocyte chemoattractant protein 1, a chemo-attractant for monocytes and basophils. This protein is also known as CCL2, C-C chemokine ligand 2. A representative amino acid and nucleotide sequence for MCP-1 is NP_002973 and NM_002982, respectively.

As used herein, “MPO” refers to myeloperoxidase, a heme protein that is a major component of azurophillic granules of neutrophils. A representative amino acid and nucleotide sequence for MPO is NP_000241 and NM_000250, respectively.

As used herein, “OPG” refers to osteoprotegerin, an osteoblast decoy receptor that acts as a negative regulator of bone resorption. This protein is also known as TNF receptor superfamily member 11B (TNFRS11B). A representative amino acid and nucleotide sequence for OPG is NP_002537 and NM_002546, respectively.

As used herein, “TIM3” refers to T-cell immunoglobulin and mucin domain containing-3, a T cell surface protein that regulates macrophage activation and promotes immunological tolerance. This protein is also known as hepatitis A viral cellular receptor 2 (HAVCR2). A representative amino acid and nucleotide sequence for TIM3 is NP_116171 and NM_032782, respectively.

As used herein, “ALDH1A1” refers to aldehyde dehydrogenase 1 family member Al, an enzyme in the alcohol metabolism pathway. A representative amino acid and nucleotide sequence for ALDH1A1 is NP_000680 and NM_000689, respectively.

As used herein, “IL-6” refers to interleukin 6, a chemokine that mediates inflammation. A representative amino acid and nucleotide sequence for 11-6 is NP_000591 and NM_000600, respectively.

As used herein, “KLK6” refers to kallikrein 6, a serine protease. A representative amino acid and nucleotide sequence for KLK-6 is NP_000416 and NM_001012964, respectively.

As used herein, “L1CAM” refers to L1 cell adhesion molecule, a cell adhesion molecule important in nervous system development. A representative amino acid and nucleotide sequence for L1CAM is NP_001012982 and NM_000425, respectively.

As used herein, “MIA” refers to melanoma inhibitory activity, a melanoma derived growth regulatory protein. A representative amino acid and nucleotide sequence for MIA is NP_001189482 and NM_001202553, respectively.

As used herein, “MDK” refers to midkine, a secreted growth factor important in angiogenesis. This protein has multiple isoforms. A representative amino acid and nucleotide sequence for MDK is NP_001012333 and NM_001012333, respectively.

As used herein, “NSE” refers to enolase, an isoenzyme found in neuronal cells. This protein is also known as ENO2. A representative amino acid and nucleotide sequence for NSE is NP_001966 and NM_001975, respectively.

As used herein, “ON (SPARC)” refers to secreted protein acidic and cysteine rich, a matrix associated protein. A representative amino acid and nucleotide sequence for SPARC is NP_003109 and NM_003118, respectively.

As used herein, “TGM2” refers to a transglutaminase, a cross linking protein involved in apoptosis. There are multiple isoforms of this protein. A representative amino acid and nucleotide sequence for TGM2 is NP_001310245 and NM_001323326, respectively.

As used herein, “TWEAK” refers to TNF superfamily member 12, a cytokine that is a ligand for TWEAK receptor. This protein is also known as TNFSF12. A representative amino acid and nucleotide sequence for TWEAK is NP_003800 and NM_003809, respectively.

As used herein, “VEGF-A” refers to vascular endothelial growth factor A, a growth factor involved in angiogenesis .There are many isoforms of this protein. A representative amino acid and nucleotide sequence for VEGF-A is NP_001020537 and NM_001025366, respectively.

As used herein, “YKL40” refers to chitinase 3 like protein, a glycol hydrolase that does not have chitinase activity. A representative amino acid and nucleotide sequence for YKL40 is NP_001267 and NM_001276, respectively.

As used herein, the term “not substantially bind” means that the detectable signal from the binding of the antibody to a component in a sample is within one or two standard deviations of the signal generated due to the presence of an unrelated polypeptide control such as bovine serum albumin.

As used herein, “specific binding” refers to an antibody that reacts or associates more frequently, more rapidly, with greater duration, with greater affinity, or with some combination of the above to an epitope or protein than with alternative substances, including unrelated proteins. In certain embodiments, “specifically binds” means, for instance, that an antibody binds to a protein with a K_{D} of about 0.1 mM or less, but more usually less than about 1 μM. In certain embodiments, “specifically binds” means that an antibody binds to a protein at times with a K_{D} of at least about 0.1 μM or less, and at other times at least about 0.01 μM or less. It is understood that an antibody or binding moiety that specifically binds to a first target may or may not specifically bind to a second target. As such, “specific binding” does not necessarily require (although it can include) exclusive binding, i.e. binding to a single target. Thus, an antibody may, in certain embodiments, specifically binds to more than one target. In certain alternative embodiments, an antibody may be bispecific and comprise at least two antigen-binding sites with differing specificities.

The term “comprising” refers to a composition, compound, formulation, or method that is inclusive and does not exclude additional elements or method steps.

The term “consisting of” refers to a compound, composition, formulation, or method that excludes the presence of any additional component or method steps.

The term “consisting essentially of” refers to a composition, compound, formulation or method that is inclusive of additional elements or method steps that do not materially affect the characteristic(s) of the composition, compound, formulation or method.

The term “isolated” refers to the separation of a material from at least one other material in a mixture or from materials that are naturally associated with the material.

As used herein, “marker” refers to any molecule, such as a gene, gene transcript (for example mRNA), polypeptide or protein or fragment thereof produced by a subject which is useful in differentiating subjects having colorectal cancer from normal or healthy subjects.

The terms “patient” or “subject” are used interchangeably and refer to any member of Kingdom Animalia. Preferably a subject is a mammal, such as a human, domesticated mammal or a livestock mammal.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” refers to a pharmaceutically-acceptable material, composition or vehicle, such as a liquid or solid filler, diluent, excipient, solvent or encapsulating material, involved in carrying or transporting the compound or analogue or derivative from one organ, or portion of the body, to another organ, or portion of the body. Each carrier must be “acceptable” in the sense of being compatible with the other ingredients of the formulation and not injurious to the patient. Some examples of materials which may serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “purified” or “to purify” or “substantially purified” refers to the removal of inactive or inhibitory components (e.g., contaminants) from a composition to the extent that 10% or less (e.g., 10%, 9%, 8%, 7%, 6%, 5%, 4%, 3%, 2%, 1% or less) of the composition is not active compounds or pharmaceutically acceptable carrier.

As used herein, the term “risk for the presence of” (e.g., at risk for, cancer, etc.) refers to a subject (e.g., a human) whose current status is that the subject has a disease state or an increased risk of the presence of the disease state such as colorectal cancer.

The “sample” may be of any suitable type and may refer, e.g., to a material in which the presence or level of markers can be detected. Preferably, the sample is obtained from the subject so that the detection of the presence and/or level of markers may be performed in vitro. Alternatively, the presence and/or level of markers can be detected in vivo. The sample can be used as obtained directly from the source or following at least one step of (partial) purification. Typically, the sample is an aqueous solution, biological fluid, cells or tissue. Preferably, the sample is blood, plasma, sweat, serum, urine, or feces.

As used herein, “sensitivity” refers to a classification function that measures the proportion of known positives in a sample set that are correctly identified as positives by the assay. For example, the percentage of sick people who are identified by the assay as having the condition.

As used herein, “specificity” refers to a classification function that measures the proportion of known negatives in the sample set that are correctly identified by the assay as not having the condition. For example, the percentage of healthy people who are correctly identified by the assay as not having the condition.

As used herein the terms “treating”, “treat” or “treatment” include administering a therapeutically effective amount of a compound sufficient to reduce or delay the onset or progression of colorectal cancer, or to reduce or eliminate at least one symptom of colorectal cancer.

Methods and Kits for Detecting the Presence of Markers in a sample

The methods and kits as described include the detection of one or more markers in a sample from a subject of unknown status. In embodiments, the detection of a combination of markers is useful to assess the presence of or the risk of the presence of cancer, to conduct an examination of the colon for cancer, and/or for administering or monitoring treatment. In embodiments, the sample comprises blood, plasma, serum, saliva, sweat, urine, or feces. In some embodiments, the sample is blood taken from a routine blood draw. The sample can be obtained as a part of a routine screening during a checkup, upon suspicion of the presence of cancer, during treatment, upon completion of treatment, and/or as a periodic follow-up following remission of the cancer. In embodiments, the cancer is colorectal cancer.

In embodiments, a method comprises detecting at least four different polypeptides and/or nucleic acids coding for the polypeptides in the sample by contacting the sample with at least four reagents, each reagent specifically detecting the presence and/or an amount of one of the at least four polypeptides and/or nucleic acids coding for the polypeptides, wherein the at least four polypeptides comprise GDF15, keratin 1-10, and two or more of hepsin, IL-8, CEA, L1CAM, MCP-1, and OPG; and determining whether the combination of the presence or detected amounts of each of the at least four different polypeptides and/or nucleic acids coding for the polypeptides is indicative of the presence of or an increased risk of the presence of colorectal cancer. In embodiments, a blood sample is obtained from the subject and the amounts of at least four different markers are detected. In embodiments, the amounts of each the at least four different polypeptides and/or nucleic acids coding for the polypeptides are analyzed with a predictive model and the presence of or the risk that the subject has colorectal cancer is assessed. In embodiments, the presence or risk of the presence of adenomatous polyps, stage I, stage II, stage III, or stage IV colorectal cancer can be determined.

In embodiments, one or more additional markers are analyzed including, without limitation, AFP, CATD, ferritin, CD44, ALDH1A1, EPCAM, FAP, Galectin 3, IL-6, kallikrein 6, keratin 6, MIA, midkine, TWEAK, NSE, ON (SPARC), TGM2, VEGFA, YKL40, and combinations thereof.

If the sample from the subject indicates the presence of or a risk of the presence of high risk adenomatous polyps or colorectal cancer, the subject can undergo an examination of the colon for cancer such as by a colonoscopy, a sigmoidoscopy, a biopsy, a CAT scan, or MRI. In embodiments, if the sample from the subject indicates the presence of or a risk of the presence of colorectal cancer, whether or not identified by additional testing, the subject can be treated with a treatment for colorectal cancer. In embodiments, a treatment regimen can be selected depending on whether the sample for the subject indicates whether the subject has adenomatous polyps or stage I colorectal cancer versus Stage III or IV colorectal cancer. Subjects having Stage III or IV colorectal cancer may receive a more aggressive treatment regimen.

In embodiments, a kit comprises at least four different reagents; each reagent specifically detecting a polypeptide and/or nucleic acid coding for the polypeptide in a sample from a subject with unknown status; and at least one standard comprising a known amount of at least one of polypeptides and/or nucleic acids coding for the polypeptides.

In embodiments, a kit comprises a computer readable medium containing instructions for analyzing the combination of the detected amount of each of the polypeptides and/or nucleic acids coding for the polypeptides from a subject with unknown status with a mathematical model to generate a risk assessment of having or not having colorectal cancer in the subject. In embodiments, the mathematical model is generated using a supervised machine learning method.

In other embodiments, a kit comprises a computer readable medium containing instructions to access a database of profiles of the combination of the detected amount of each of the polypeptides and/or nucleic acids coding for the polypeptides from subjects having stage I colorectal cancer, stage II colorectal cancer, stage III colorectal cancer, stage IV colorectal cancer, high risk adenomatous polyps, low risk adenomatous polyps, and/or normal subjects; and to determine whether the profile from the subject with unknown status is similar to any of the profiles from subjects with known status to identify whether the subject with unknown status has stage I colorectal cancer, stage II colorectal cancer, stage III colorectal cancer, stage IV colorectal cancer, high risk adenomatous polyps, low risk adenomatous polyps, or is normal.

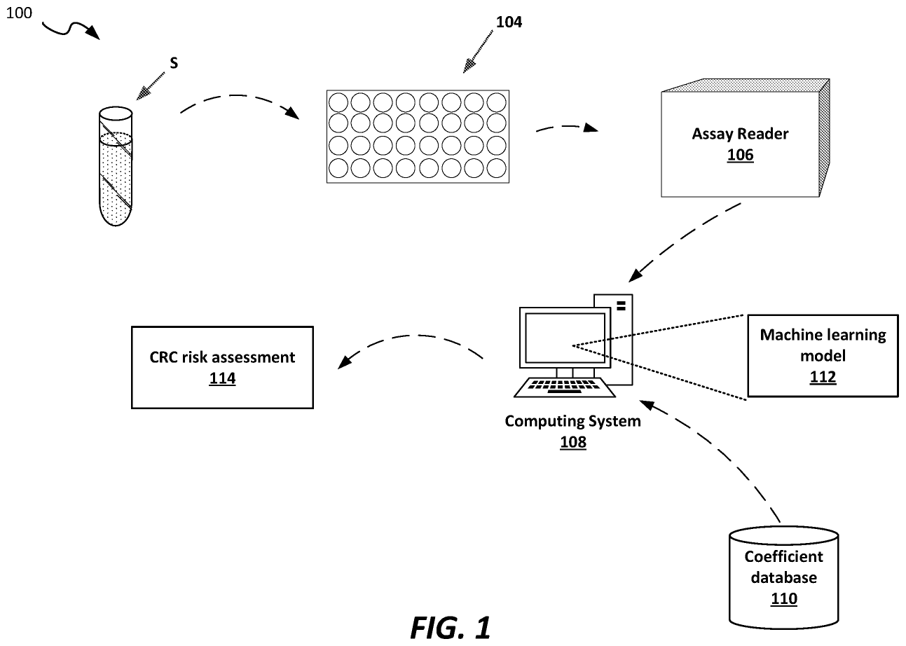

FIG. 1 illustrates a schematic diagram of a kit 100 for detecting at least four different polypeptides in a sample S from a subject with unknown status. The sample S is applied to a solid surface 104 having at least four reagents attached that specifically bind to one of a plurality of polypeptides in the sample S. The plurality of polypeptides comprise GDF15, keratin 1-10 and two or more of hepsin, IL-8, CEA, L1CAM, MCP-1, and OPG. At least four detectably labelled secondary reagents are included having different labels to distinguish between the at least four different polypeptides.

The solid surface 104 including the reagents and polypeptides is read with an assay reader 106 to measure the amount of each polypeptide in the sample S. The amounts are communicated to a computing system 108 along with coefficients for each of the detected amount of each of the polypeptides. The coefficients are retrieved from a coefficient database 110. Each of the detected amounts of the polypeptides are multiplied by their corresponding coefficient to generate a weighted level for each of the polypeptides. The combination of weighted levels for each polypeptide is then analyzed using a machine learning model 112 to determine a risk assessment for the subject having colorectal cancer based on a change or lack thereof from the weighted values of the polypeptides for normal subjects.

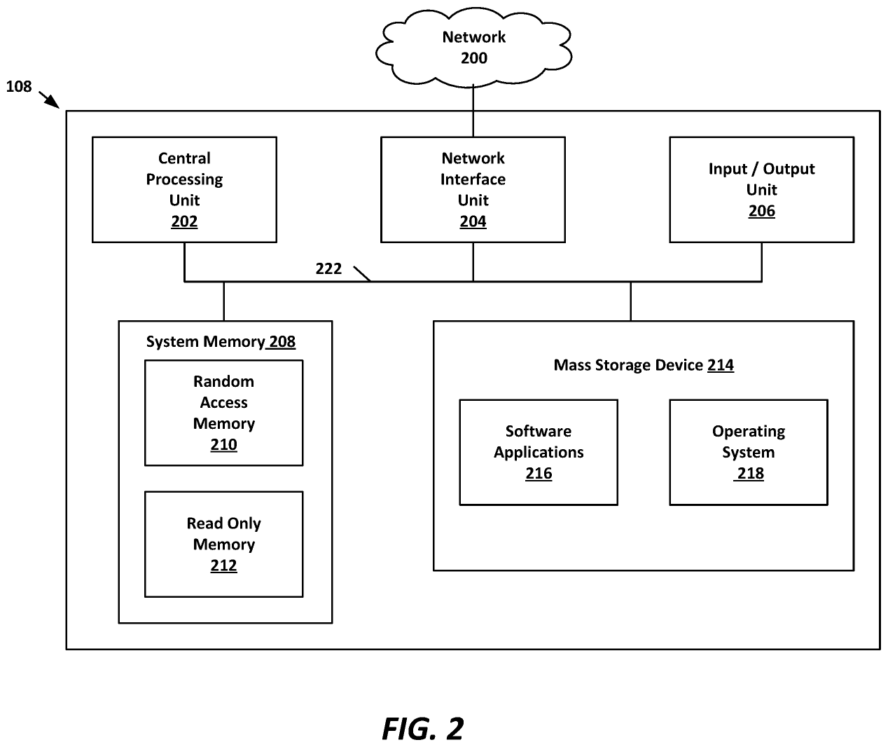

FIG. 2 is a block diagram illustrating an example of the physical components of the computing device 108. In the example shown in FIG. 2, the computing device 108 includes at least one central processing unit (“CPU”) 202, a system memory 208, and a system bus 222 that couples the system memory 208 to the CPU 202. The system memory 208 includes a random access memory (“RAM”) 210 and a read-only memory (“ROM”) 212. A basic input/output system that contains the basic routines that help to transfer information between elements within the computing device 108, such as during startup, is stored in the ROM 212. The computing device 108 further includes a mass storage device 214. The mass storage device 214 is able to store software instructions and data such as machine learning models.

The mass storage device 214 is connected to the CPU 202 through a mass storage controller (not shown) connected to the system bus 222. The mass storage device 214 and its associated computer-readable storage media provide non-volatile, non-transitory data storage for the computing device 108. Although the description of computer-readable storage media contained herein refers to a mass storage device, such as a hard disk or solid state disk, it should be appreciated by those skilled in the art that computer-readable data storage media can include any available tangible, physical device or article of manufacture from which the CPU 202 can read data and/or instructions. In certain embodiments, the computer-readable storage media comprises entirely non-transitory media.

Computer-readable storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the computing device 108.

According to various embodiments, the computing device 108 can operate in a networked environment using logical connections to remote network devices through a network 200, such as a wireless network, the Internet, or another type of network. The computing device 108 may connect to the network 200 through a network interface unit 204 connected to the system bus 222. It should be appreciated that the network interface unit 204 may also be utilized to connect to other types of networks and remote computing systems. The computing device 108 also includes an input/output controller 206 for receiving and processing input from a number of other devices, including a touch user interface display screen, or another type of input device Similarly, the input/output controller 206 may provide output to a touch user interface display screen or other type of output device.

As mentioned briefly above, the mass storage device 214 and the RAM 210 of the computing device 108 can store software instructions and data. The software instructions include an operating system 218 suitable for controlling the operation of the computing device 108. The mass storage device 214 and/or the RAM 210 also store software instructions, that when executed by the CPU 202, cause the computing device 108 to provide the functionality discussed in this document. For example, the mass storage device 214 and/or the RAM 210 can store software instructions that, when executed by the CPU 202, cause the computing device 108 to assess a subject's risk of having CRC.

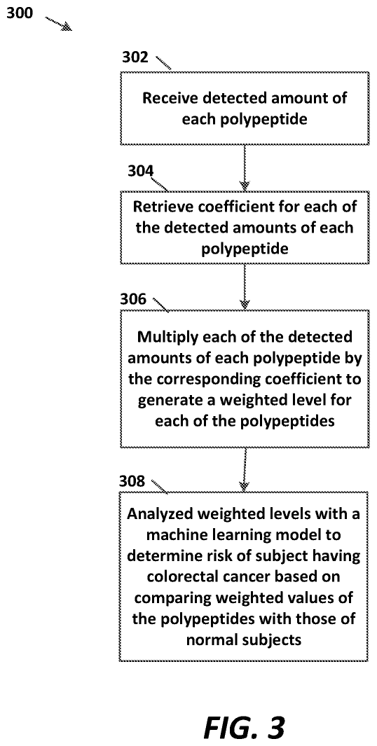

FIG. 3 is a flow chart illustrating an example method 300 of detecting at least four different polypeptides in a sample from a subject with unknown status. In some embodiments, the method 300 is performed by the computing device 108 of FIGS. 1 and 2.

At operation 302, a detected amount of each polypeptide is received at the computing device 108. In some embodiments, the detected amount is received from an assay reader 106 such as MAGPIX.

At operation 304, a coefficient for each of the detected amounts of each polypeptide is retrieved. In some embodiments, the coefficient is retrieved from a coefficient database 110 by the computing device 108.

At operation 306, each of the detected amounts of the polypeptides is multiplied by the corresponding coefficient to generate a weighted level for each of the polypeptides. In some embodiments, the computing device 108 performs this calculation using the information received from the assay reader 106 and coefficient database 110.

At operation 308, the combination of weighted levels is analyzed for each polypeptide using a machine learning model. This analysis determines a probability that a subject has colorectal cancer based on comparing the weighted levels to those of normal subjects. In some embodiments, the computing device 108 performs this analysis and outputs a risk assessment for a subject.

### Methods

This disclosure describes methods for detecting the amounts of or the presence of at least four different markers in combination and determining whether the combination of the detected amounts or presence of the at least four polypeptides and/or nucleic acids coding for the polypeptides is indicative of the presence of or an increased risk of the presence of colorectal cancer. In embodiments, a method for detecting at least four markers in a sample from a subject with unknown status comprises: detecting the presence and/or an amount of at least four polypeptides and/or nucleic acids coding for the polypeptides in the sample by contacting the sample with at least four reagents, each reagent specifically binding to one of the at least four polypeptides and/or nucleic acids coding for the polypeptides, wherein the at least four polypeptides comprise GDF15, keratin 1-10, and two or more of hepsin, IL-8, CEA, L1CAM, MCP-1, and OPG; and determining whether the combination of the presence of and/or detected amounts of each of the at least four polypeptides and/or nucleic acids coding for the polypeptides is indicative of the presence of or an increased risk of the presence of colorectal cancer in the subject. In embodiments, the method comprises detecting no more than 4, 5, 6, 7, 8, 9, 10, 11, 12, 13, 14, 15, 16, 17, 18, 19, 20, 21,22, 23, 24, 25, 26, 27, 28, 29, 30, 31, 32, 33, 34, 35, 36, 37, 38, 39, 40, 41, 42, 43, 44, 45, 46, 47, 48, 49, or 50 polypeptides or nucleic acids coding for the polypeptides.

In other embodiments, a method for conducting an examination of the colon for colorectal cancer in a subject comprises: detecting the presence and/or an amount of at least four polypeptides and/or nucleic acids coding for the polypeptides in the sample by contacting the sample with at least four reagents, each reagent specifically binding to one of the at least four polypeptides and/or nucleic acids coding for the polypeptides, wherein the at least four polypeptides comprise GDF15, keratin 1-10, and two or more of hepsin, IL-8, CEA, L1CAM, MCP-1, and OPG; determining whether the combination of the presence of and/or detected amounts of the at least four polypeptides and/or nucleic acids coding for the polypeptides is indicative of the presence of or the increased risk of the presence of colorectal cancer in the subject; and if the subject has the presence of or an increased risk of the presence of colorectal cancer, conducting an examination of the colon. In embodiments, the colon is examined by a method comprising a colonoscopy, a virtual colonoscopy, a sigmoidoscopy, a biopsy, a CAT scan, a MRI, or combinations thereof.

In other embodiments, a method for treating colorectal cancer in a subject comprises: detecting the presence and/or an amount of at least four polypeptides and/or nucleic acids coding for the polypeptides in the sample by contacting the sample with at least four reagents, each reagent specifically binding to one of the at least four polypeptides and/or nucleic acids coding for the polypeptides, wherein the at least four polypeptides comprise GDF15, keratin 1-10, and two or more of hepsin, IL-8, CEA, L1CAM, MCP-1, and OPG; determining whether the combination of the presence of and/or detected amounts of each of the at least four polypeptides and/or nucleic acids coding for the polypeptides is indicative of the presence of or an increased risk of the presence of colorectal cancer in the subject; and if the subject has the presence of or an increased risk of the presence of colorectal cancer, treating the subject with a treatment effective for colorectal cancer. In embodiments, the treatments effective for colorectal cancer comprise surgery, chemotherapy, and combinations thereof. Chemotherapy agents comprise 5-fluorouracil, folinic acid, oxaplatin, irinotecan, capecitabine, inhibitors of VEGF, trypsin kinase inhibitors, inhibitors of EGFR, anti-VEGF antibodies, human VEGF receptor fusion proteins, anti-VEGF receptors antibodies, anti-EGFR antibodies, checkpoint inhibitors, anti-PD-1 antibodies, and anti-PD-L1 antibodies, or combinations thereof. In embodiments, a treatment regimen can be selected to be more aggressive depending on whether the subject with unknown status is identified as having stage III or IV colorectal cancer. For example, a subject with adenomatous polyps, or stage I colorectal cancer can be treated with surgery to remove the tumor or parts of the colon. For stage II and stage III colorectal cancer, surgery with chemotherapy agents such as 5-fluorouracil, folinic acid, oxaplatin, irinotecan, capecitabine, or combinations thereof. For stage IV colorectal cancer, chemotherapy is administered before and after surgery and includes both targeted agents such as inhibitors of VEGF and one or more of 5-fluorouracil, folinic acid, oxaplatin, irinotecan, capecitabine.

In embodiments, the at least four polypeptides comprise GDF15, MCP-1, IL-8 and keratin 1-10. In other embodiments, the at least four polypeptides comprise GDF15, MPO, IL-8 and keratin 1-10. In yet other embodiments, the at least four polypeptides comprise GDF15, CATD, IL-8 and keratin 1-10. In further embodiments, the at least four polypeptides comprise GDF15, ferritin, IL-8 and keratin 1-10.

In some embodiments, the at least four polypeptides comprise GDF15, hepsin, IL-8, and keratin 1-10. In some embodiments, the at least four polypeptides comprise GDF15, keratin 1-10, CEA, L1CAM, MCP-1, and OPG. In some embodiments, the at least four polypeptides comprise GDF15, keratin 1-10, CEA, L1CAM, MCP-1, OPG, hepsin, and IL-8. In some embodiments, the at least four polypeptides comprise GDF15, keratin 1-10, CEA, L1CAM, hepsin, IL-8, AFP, CATD, CD44, ferritin, MIA, MDK, NSE, ON (SPARC), TWEAK, and YKL40. In some embodiments, the at least four polypeptides comprise GDF15, keratin 1-10, CEA, L1CAM, MCP-1, OPG, hepsin, IL-8, AFP, CATD, CD44, ferritin, MIA, MDK, NSE, ON (SPARC), TWEAK, and YKL40.

In embodiments, the methods further comprise obtaining the sample from the subject, the subject having an unknown status. In embodiments, the sample comprises blood, plasma, serum, sweat, saliva, urine, tissue, or feces. In embodiments, the sample is retrieved in a blood draw. In embodiments, the sample comprises circulating tumor cells, circulating tumor nucleic acids, exosomes, methylated DNA, or combinations thereof. The sample can be obtained as a part of a routine screening of a health checkup, upon suspicion of the presence of cancer, during treatment, upon completion of treatment, and as periodic follow-up following remission of the cancer. In some embodiments, the sample is processed to remove cells, particulate matter, and/or other contaminants. In embodiments, the sample can be processed to concentrate polypeptide components.

In embodiments, the methods described herein can also include detecting the presence of or an amount of one or more additional polypeptides or nucleic acids encoding polypeptides. The one or more additional polypeptides comprise AFP, ferritin, CATD, CD44, ALDH1A1, EPCAM, FAP, Galectin 3, IL-6, kallikrein 6, CEA, keratin 6, L1CAM, MIA, midkine, TWEAK, NSE, ON (SPARC), TGM2, VEGFA, YKL40, or combinations thereof.

In embodiments, the methods described herein comprise detecting the presence and/or an amount of at least eight polypeptides and/or nucleic acids coding for the polypeptides in the sample by contacting the sample with at least eight reagents, each reagent specifically binding to one of the at least eight polypeptides and/or nucleic acids coding for the polypeptides, wherein the at least eight polypeptides comprise AFP, ferritin, CATD, CD44, GDF15, hepsin, IL-8, and keratin 1-10; and determining whether the combination of the presence of and/or detected amounts of each of the at least eight polypeptides and/or nucleic acids coding for the polypeptides is indicative of the presence of or an increased risk of the presence of colorectal cancer in the subject. In embodiments, the method comprises detecting no more than 8, 9, 10, 11, 12, 13, 14, 15, 16, 17, 18, 19, 20, 21,22, 23, 24, 25, 26, 27, 28, 29, 30, 31, 32, 33, 34, 35, 36, 37, 38, 39, 40, 41, 42, 43, 44, 45, 46, 47, 48, 49, or 50 polypeptides or nucleic acids coding for the polypeptides.

In embodiments, the methods described herein comprise detecting the presence and/or an amount of at least sixteen polypeptides and/or nucleic acids coding for the polypeptides in the sample by contacting the sample with at least sixteen reagents, each reagent specifically binding to one of the at least sixteen polypeptides and/or nucleic acids coding for the polypeptides, wherein the at least sixteen polypeptides comprise AFP, ferritin, CATD, CD44, GDF15, hepsin, IL-8, keratin 1-10, L1CAM, MIA, MDK, NSE, ON (SPARC), TWEAK,YKL40, and CEA; and determining whether the combination of the presence of and/or detected amounts of each of the at least sixteen polypeptides and/or nucleic acids coding for the polypeptides is indicative of the presence of or an increased risk of the presence of colorectal cancer in the subject. In embodiments, the method comprises detecting no more than 16, 17, 18, 19, 20, 21,22, 23, 24, 25, 26, 27, 28, 29, 30, 31, 32, 33, 34, 35, 36, 37, 38, 39, 40, 41, 42, 43, 44, 45, 46, 47, 48, 49, or 50 polypeptides or nucleic acids coding for the polypeptides.

In embodiments, the at least four reagents comprise one or more primary antibodies or antigen binding fragments, each primary antibody or antigen binding fragment specifically binds to one of the polypeptides. In some embodiments, each of the at least four reagents are primary antibodies or antigen binding fragments, each reagent specifically binds to a different one of the identified polypeptides. In other embodiments, the methods comprise additional primary antibody or antigen binding fragments thereof that specifically bind to a polypeptide comprising AFP, ferritin, CATD, CD44, ALDH1A1, EPCAM, FAP, Galectin 3, IL-6, kallikrein 6, CEA, keratin 6, L1CAM, MIA, MDK, TWEAK, NSE, ON (SPARC), TGM2, VEGFA, or YKL40. In some embodiments, each of the additional reagents are primary antibodies or antigen binding fragments, each reagent binds to a different one of the identified additional polypeptides.

Antibodies or antigen binding fragments can be prepared using standard techniques. The sequences of each of the polypeptides described herein have been described in publicly available databases as identified herein. In some cases, the polypeptides have multiple isoforms. In embodiments, an antibody is selected that binds to all of the isoforms. In other embodiments, and antibody is selected that specifically binds to a single isoform and does not substantially bind to other isoforms. For example, an antibody that specifically binds to all isoforms of CD44 binds to epitope 1 on CD44. In other embodiments, an antibody is selected that binds to isoform CD44 variant 6.

In embodiments, an antibody is selected that specifically binds to one of the identified polypeptides or additional polypeptides, and does not substantially bind to any other of the identified polypeptides or additional polypeptides. In embodiments, it is preferred that the antibodies have an affinity for the polypeptide of 10⁻⁷ to 10⁻¹² K_{D}. In other embodiments, it is preferred that the antibody or antigen binding fragment thereof can detect a range of concentrations of the polypeptides, preferably detecting at least 0.01 picograms/ml. In embodiments, each antibody or antigen binding fragment that specifically binds to a polypeptide, binds to the polypeptide with a percent of coefficient of variation of 20%, 15%, 10%, 5%, 4%, 3%, 2%, 1%, or less.

In some embodiments, the at least four reagents comprise a reagent that specifically binds to a nucleic acid coding for one of the at least four polypeptides, wherein the at least four polypeptides comprise GDF15, keratin 1-10 and two or more of hepsin, IL-8, CEA, L1CAM, MCP-1, and OPG. In embodiments, the reagent comprises a set of primers, a probe, an aptamer, and combinations thereof.

In embodiments, each of the at least four reagents are attached to a solid surface. In embodiments, each of the reagents is attached to a different solid surface or a different location on a solid surface. In embodiments, the solid surface comprises a bead, a magnetic bead, and a slide, a well of a multiwell plate, a chip, a microfluidic channel or combinations thereof. In embodiments, each reagent is attached to a different solid surface, each of the different solid surfaces having a different internal marker. In some embodiments, each of the different solid surfaces are the same type of solid surface and differ from one another based on a different internal marker. In embodiments, the different internal marker comprises a radioactive isotope tag, a quantum dot, a protein or peptide tag, an RFID tag, or a fluorescent dye. In embodiments each reagent is attached to a bead having a unique and different internal marker so that the presence or amount of each of polypeptides detected by the reagents is separately identifiable by the presence of the internal marker.

In embodiments, the sample is contacted with at least four reagents. Each reagent can be contacted with the sample in a separate container or various combinations of reagents can be combined in one or more containers. In embodiments, the sample and the at least four reagents are contacted in a single container. In embodiments, the container comprises a well of a multiwell plate, a tube, a microfluidic channel, a slide, or a sample port. In some embodiments, each reagent is present in the mixture at a similar concentration as the other reagents.

In embodiments, once the sample is contacted with at least four reagents, each of the polypeptides or nucleic acids coding for the polypeptides if present in the sample form a complex with its specific reagent. Complexes are washed and then detected using a detectably labelled secondary reagent. In embodiments, the methods further comprise contacting the sample with at least four detectably labelled secondary reagents, each detectably labelled secondary reagent specifically binds to or detects one of the at least four polypeptides or nucleic acids coding for the polypeptides; and each of the at least four detectably labelled secondary reagents has a different detectable label. In embodiments, each of the detectably labelled secondary reagents has a detectable label different from the other detectably labelled secondary reagents. In embodiments, the secondary reagent is labelled with a fluorescent dye, a radiolabel, a protein or peptide tag, an enzyme, or a luminescent reactant. In embodiments, the label on the secondary reagent is different than the internal label on the solid surface.

In embodiments, one or more secondary detectably labelled reagents, can be added, wherein each of the detectably labelled secondary reagents binds to or detects one of the additional polypeptides and/or nucleic acids coding for the additional polypeptides, the additional polypeptides comprising AFP, ferritin, MCP-1, OPG, CATD, CD44, ALDH1A1, EPCAM, FAP, Galectin 3, IL-6, kallikrein 6, CEA, keratin 6, L1CAM, MIA, MDK, TWEAK, NSE, ON (SPARC), TGM2, VEGFA, or YKL40.

In embodiments, the detectably labelled secondary reagent, is a secondary antibody or antigen binding fragment thereof that specifically binds one of the at least four polypeptides comprising GDF15, hepsin, IL-8, and keratin 1-10. In embodiments, additional secondary antibody or antigen binding fragment thereof specifically binds to one of the additional polypeptides, the additional polypeptides comprising AFP, ferritin, MCP-1, OPG, CATD, CD44, ALDH1A1, EPCAM, FAP, Galectin 3, IL-6, kallikrein 6, CEA, keratin 6, L1CAM, MIA, midkine, TWEAK, NSE, ON (SPARC), TGM2, VEGFA, or YKL40. In embodiments, the detectably labelled secondary antibody or antigen binding fragment thereof binds to a different epitope on the polypeptide than the primary antibody or antigen binding fragment thereof that binds to the same polypeptide. In embodiments, each of the at least four detectably labelled secondary reagents, are antibodies or antigen binding fragments, each antibody or antigen binding fragments thereof specifically binds to one of the at least four polypeptides.

In embodiments, the sample is then analyzed to detect the presence and/or amount of each of the at least four polypeptides and/or nucleic acids coding for the polypeptides. In some embodiments, the internal marker of the solid surface is detected using fluorescent activated cell sorting, using absorption profiles at different wavelengths depending on the internal marker, detecting different quantum dots, using binding to a specific protein or peptide tag, and/or measuring different radioactive isotopes. In embodiments, detecting the internal marker, identifies which one of the at least four polypeptides or nucleic acids coding for the at least four polypeptides is being detected. The label on the secondary labelled reagent is then detected using fluorescent activated cell sorting, using absorption profiles at different wavelengths depending on the internal marker, using binding to a specific protein or peptide tag, measuring enzyme activity, measuring luminescent activity, and/or measuring different radioactive isotopes. In embodiments, the internal marker of the solid surface and the secondary labelled reagent are different from one another.

An amount of each of the polypeptides or nucleic acids encoding the polypeptide can be determined using a standard curve. In embodiments, at least one standard comprises a known amount of one or more of each of the at least four polypeptides or nucleic acids coding for the polypeptide. In some embodiments, each standard contains a different concentration of the polypeptide or nucleic acid coding for the polypeptide. In embodiments, the standard contains all of the polypeptides being detected in the assay. In embodiments, the standard is provided in lyophilized form and instructions are provided for appropriate dilution. In embodiments, a set of standards includes a range of concentrations from 0.01 picograms to 1 ng.

In embodiments, a standard control is a low concentration quality control standard for each of the at least four polypeptides or nucleic acids coding for the polypeptides. In embodiments, a standard control is a high concentration quality control standard for each of the at least four polypeptides or nucleic acids coding for the polypeptides.

In embodiments, a standard can be a low concentration quality control sample, and/or a high concentration quality control standard. In some embodiments, the concentration of the polypeptide in the low quality control standard ranges from 0.001 to 500 ng/ml depending on the polypeptide. For example, a low concentration quality control standard for GDF15 is about 0.01 to about 0.5 ng/ml, a low concentration quality control standard for hepsin is about 2 to about 10 ng/ml, a low concentration quality control standard for IL-8 is about 0.1 to about 0.5 ng/ml, and a low concentration quality control standard for keratin 1-10 is about 100 to about 500 ng/ml.

In some embodiments, the concentration of the polypeptide in the high quality control standard ranges from 0.05 to 5000 ng/ml depending on the polypeptide. For example, a high concentration quality control standard for GDF15 is about 1 to about 5 ng/ml, a high concentration quality control standard for hepsin is about 20 to about 50 ng/ml, a high concentration quality control standard for IL-8 is about 0.1 to about 1 ng/ml, and a high concentration quality control standard for keratin 1-10 is about 1000 to about 5000 ng/ml.

In embodiments, control samples are analyzed in a similar manner as to the samples from the subject. Control samples include a sample or pooled samples from a subject known to have stage I colorectal cancer, a sample from a subject known to have stage II colorectal cancer, a sample from a subject known to have stage III colorectal cancer, a sample from a subject known to have stage IV colorectal cancer, a sample from a subject known to not have colorectal cancer, a sample from a subject having a low risk adenomatous polyps, a sample from a subject having a high risk adenomatous polyps, and combinations thereof.

In a certain embodiment, serum samples are diluted in assay buffer and standards and controls are diluted in serum matrix. Samples, standards (blank and 7 dilutions of standard), and controls (low and high) are combined with a mixture of color-coded solid surfaces (e.g. microspheres) coated with primary antibodies, each primary antibody coated on a solid surface with a different color internal marker, in 96 well or 384 well plates in duplicate wells. Each assay well contains about 100-300 microspheres for each marker, and the mixture is incubated 18-20 hours. The microspheres are washed. A mixture of biotinylated secondary antibodies targeting all markers are added to each well, and incubated for 1 hour. Next, streptavidin-phycoerythrin is added to each well without decanting the secondary detection antibodies, and incubated for 30 minutes. The microspheres are washed and resuspended with wash buffer and run on Luminex® 200™, HTS, FLEXMAP 3D® or MAGPIX® with xPONENT® software. The raw data is exported (automatically or manually) to analysis software for quantification and scoring. Quantitative analysis of samples and quality controls are calculated based on a standard curve of known concentration for each marker. The assay performance is qualified by both the low and high quality control concentrations falling within expected ranges for their specific lots for each marker. Low and high quality control values are chosen based average serum ranges detected in the assay for each marker. The calculated marker concentrations for each sample is further analyzed by the machine learning algorithm in order to determine the probability of the presence of disease.

In embodiments, once the presence and/or detected amount of the at least four polypeptides in the sample is obtained, whether the combination of the presence of and/or detected amounts of each of the at least four polypeptides and/or nucleic acids coding for the polypeptides is indicative of the presence of or an increased risk of the presence of colorectal cancer in the subject is determined.

In embodiments, a method further comprises determining the accuracy of the measurement of the detected amounts of each of the polypeptides and/or nucleic acids by determining the percent coefficient of variation (% CV) for each of the polypeptides and/or nucleic acids coding for the polypeptide based on measurement of the standard for each of the polypeptides and/or nucleic acids coding for the polypeptides. In embodiments, the % CV of the measurement is 20%, 15%, 10%, 5%, 4%, 3%, 2%, 1% or less.

In embodiments, the determination of the status of the subject as having or not having cancer can be made by analyzing the profile of the combination of the detected amounts of the at least four polypeptides in the sample from a subject with unknown status with a database of profiles of the combinations of the detected amounts of the at least four polypeptides from subjects known to have a low risk adenomatous polyps, a high risk adenomatous polyps, stage I, stage II, stage III, stage IV colorectal cancer, and from subjects known not to have colorectal cancer. A determination of whether the profile from the subject with an unknown profile is more similar to the profiles of those known to have colorectal cancer is indicative of the presence of colorectal cancer in the subject with an unknown status.

Alternatively, the presence of and/or detected amounts of the at least four polypeptides in the sample can be analyzed using a mathematical model to determine a risk that the subject with an unknown status has colorectal cancer. In embodiments, the mathematical model is generated by a supervised machine learning method. In some embodiments, biometric markers can be included such as height; Weight; BMI, Body Mass Index=(weight in kilograms/height in meters)/height in meters; Gender; Smoking status (nonsmoker, smoker, or ex-smoker); Alcohol consumption per week (0, 1-7, 8-14, 15-21, >21; and History of previous cancer (yes or no).

In embodiments, predictions for an individual's disease state are made using a supervised machine learning (SML) algorithm. SML models seek to map a set of measured features to a specified label. In specific embodiments, biomarker concentrations in serum serve as features used to make the prediction. The disease state for cancer in each subject is the label to be predicted by the algorithm. In other embodiments, each subject serves as an observation that will be analyzed by the SML algorithm. In yet other embodiments, unsupervised machine learning can be employed. Unsupervised ML differs from SML in that there is no pre-measured label to predict.

In embodiments, in step 1, biomarker concentrations are measured for each subject and are associated with an externally validated label, i.e. the subject's CRC diagnosis. Step 2 consists of randomly assigning subject data to subsets to be used for training or testing by the SML algorithm. Optionally, a third subset of subject data can be supplied to the algorithm for validation. In step 3, subject data from the training set is cleaned and transformed to improve algorithmic efficiency. Common data transformations include scaling, normalization, binning, and feature ratio formation. Furthermore, unsupervised ML algorithms may be used to create lower dimensional features or observation clusters that can be fed to the SML when predicting the subject's CRC state.

In embodiments, in step 4, following feature engineering, the transformed biomarker data is fed to the SML algorithm for training. During this process, the quality of label prediction is quantified using a cost function. Training includes optimizing the parameters of the cost function to improve predictive power. For model based SML, such as logistic regression and support vector machines (SVM), optimized parameters frequently take the form of numerical weights. A common cost function for this SML subclass is the log loss function. For instance-based learning, classification rules commonly serve as the optimized parameters. Examples of optimized rules include the number of nearest neighbors used in k-nearest neighbors or the biomarker concentration that demarcates CRC-positive from CRC-negative patients in binary decision trees/random forest classifiers. An alternative cost function to log loss function used in binary decision trees/random forest classifier is the gini-index. In step 5, following parameter optimization, the performance of each trained SML algorithm must be evaluated on data external to the training set. To compare alternative SML algorithms, external validation data or an appropriate resampling method is used to calculate values such as accuracy, precision, recall/sensitivity, specificity, etc. If training fails to produce an algorithm with sufficient predictive power, the process returns to step 3 for additional feature engineering and retraining. Upon choosing a trained algorithm for use, model performance is evaluated using the external test data (step 6). Test data consists of subject biomarker concentrations and cancer disease states that have not been used in SML algorithm training or validation. Provided that the predictive power measured in step 6 is sufficient, the CRC disease state can be estimated using biomarker concentrations from serum of patients with confirmed diagnosis (step 7).

In embodiments, different mathematical models can be employed. In embodiments, Model 1 uses the Universal Process Classification algorithm, a variant of K-Nearest Neighbors classification that utilizes a distance measurement to identify nearest neighbors. In embodiments, Model 2 uses support vector classifiers with radial basis function kernels during identification. In embodiments, Model 3 is a random forest classifier, an algorithm class that makes predictions by averaging the results of multiple randomly-initialized binary decision trees. In embodiments, Model 4 is also a random forest classifier.

In embodiments, a K-nearest neighbor classifier predicts that a subject has the same label as the majority of its k-nearest neighbors, where k is a positive integer. Neighbors are determined by measuring the distance between the features created during the feature engineering step of ML (step 3). The k-observations with the shortest distances are selected as neighbors. While common distance measurements include Euclidian, Manhattan, and cosine distances, any measurement that satisfy the triangle inequality can be utilized. By varying engineered features, the number of nearest neighbors (k), and distance measured, k-nearest neighbors can take a variety of forms during classification. The log loss function is a common cost function for this classifier.

In embodiments, support vector classifiers (SVC) provide a linear decision boundary in feature space that separates observations based on their labels. Observations on one side of the line are predicted to be positive while observations on the other side of the line are negative. To improve predictive capacity, SVC uses feature engineering to transform and combine features to generate a higher-dimensional feature space. An example of this is squaring the concentration of the measured biomarkers. When combined, for example, the original 8 biomarkers, there are 16 features in total. This increases the dimension of the feature space from 8 to 16 and potentially increases the distance between observed data points. This can lead to improved placement of the linear decision boundary. SVC commonly uses the log loss function with the addition of a term that acts to increase the border between the decision boundary and observed data.

In embodiments, a random forest classifier is an ensemble algorithm that averages the predictions made by multiple binary decisions trees. Binary decision trees make predictions by learning rules that segregate observations into increasingly homogeneous subgroups based on measured label values. In our case, rules include feature threshold values. Subjects with features above the threshold are partitioned into one subgroup while those below are partitioned into a separate subgroup. By using a hierarchal set of rules, nonlinear relationships can be used to make label predictions. Common cost functions used in binary decision trees/random forest include the cross-entropy function and the gini-index.

In certain embodiments, whether the presence and/or amount of each of the at least four different polypeptides in the unknown sample is indicative of an increased risk of the presence of colorectal cancer is analyzed with a computer implemented method. The computer implemented method comprising: receiving the detected amount of each of the polypeptides and/or nucleic acids coding for the polypeptides on a computing device; retrieving a coefficient for each of the detected amounts of each of the polypeptides and/or nucleic acids coding for the polypeptides from a database on the computing device; multiplying each of the detected amounts by the corresponding coefficient to generate a weighted level for each of the polypeptides on the computing device; analyzing the combination of weighted levels for each polypeptide with a model on the computing device to determine if the subject has an increased risk of colorectal cancer based on a change or lack thereof in the combination of weighted levels for each of the polypeptides detected in the sample from the subject with unknown status to the combination of predetermined weighted values of the polypeptides for normal subjects.

In embodiments, the methods described herein have a sensitivity of at least 80%, 85%, 90%, 91%, 92%, 93%, 94%, 95%, or greater. In embodiments, methods described herein, have a specificity of at least 50%, 55%, 60%, 65%, 70%, 75%, 80%, 85%, 90%, or greater. In some embodiments, a method as described herein has a sensitivity to early stage cancer of at least 90% or greater; a specificity for healthy normal of at least 50% or greater; and an Area under the Curve (AUC) of at least 80% or greater. In embodiments, in a method of conducting an examination of the colon for colorectal cancer in a subject, an examination of the colon is conducted if the sample from the subject indicates the presence of or the risk of the presence of high risk adenomatous polyps or colorectal cancer. In embodiments, examination of the colon is conducted by a colonoscopy, a virtual colonoscopy, sigmoidoscopy, a biopsy, a CAT scan, a MRI, or combinations thereof. In embodiments, if the sample from the subject indicates the presence of or the risk of the presence of colorectal cancer, whether or not identified by additional testing, the subject can be treated with a therapeutic regimen that treats colorectal cancer. Therapeutic regimens can include surgery with or without chemotherapy.

Therapeutic agents for treating colorectal cancer include 5-fluorouracil, folinic acid, oxaplatin, irinotecan. Capecitabine, inhibitors of VEGF, trypsin kinase inhibitors, inhibitors of EGFR, anti-VEGF antibodies, human VEGF receptor fusion proteins, anti-VEGF receptors antibodies, anti-EGFR antibodies, checkpoint inhibitors, anti-PD-1 antibodies, and anti-PD-L1 antibodies. Efficacy of treatment can be monitored using the methods and kits as described herein. In embodiments, a treatment regimen can be selected to be more aggressive depending on whether the subject with unknown status is identified as having stage III or IV colorectal cancer. For example, a subject with adenomatous polyps, or stage I colorectal cancer can be treated with surgery to remove the tumor or parts of the colon. For stage II and stage III colorectal cancer, subjects can be treated with surgery followed b chemotherapy agents such as 5-fluorouracil, folinic acid, oxaplatin, irinotecan, capecitabine, or combinations thereof. For stage IV colorectal cancer, chemo therapy is administered before and after surgery and includes both targeted agents such as inhibitors of VEGF, and one or more of 5-fluorouracil, folinic acid, oxaplatin, irinotecan, capecitabine.

### Kits

Another aspect of the disclosure includes kits for detecting one or more markers in a sample from a subject with an unknown status. The kits are useful to determine the presence of or the increased risk of the presence of cancer such as colorectal cancer. In embodiments, a number of different markers are detected in a sample from the subject and the combination of the markers detected is predictive of the presence or the risk of the presence of colorectal cancer. In embodiments, a detection of a combination of markers is useful in methods of examination of the colon for colorectal cancer and/or for treating colorectal cancer. In embodiments, a kit comprises at least four reagents; each reagent specifically detecting or binding to at least one polypeptide and/or nucleic acid coding for the polypeptide in a sample from the subject, wherein the polypeptides comprise GDF15, keratin 1-10, and two or more of hepsin, IL-8, CEA, L1CAM, MCP-1, and OPG; and at least one standard comprising a known amount of at least one of the polypeptides and/or nucleic acids coding for the polypeptides.

In other embodiments, the kit further comprises a computer readable medium comprising instructions for analyzing the combination of the detected amount of each of the polypeptides and/or nucleic acids coding for the polypeptide with a mathematical model to generate a risk assessment of the subject having or not having colorectal cancer. In embodiments, the mathematical model is obtained using a supervised machine learning algorithm. In certain embodiments, the supervised machine learning algorithm is a random forest classifier, support vector classifier (SVC), and an adaptation of the k-nearest neighbor's classifier.

In embodiments, the results of the mathematical model of the detected amounts of the polypeptides and/or nucleic acids coding for the polypeptides from the sample from a subject with an unknown status are analyzed for a degree of similarity to a stored representative mathematical model from samples from subjects known to have high risk adenomatous polyps, low risk adenomatous polyps, stage I colorectal cancer, stage II colorectal cancer, stage III colorectal cancer, stage IV colorectal cancer, and subjects known not to have colorectal cancer or polyps. In some embodiments, a risk assessment is made by determining how similar the mathematical model from the subject with the unknown status is to each of the stored mathematical models.

In any of the methods described herein, the subjects can be stratified into having a risk of one of high risk adenomatous polyps, low risk adenomatous polyps, stage I colorectal cancer, stage II colorectal cancer, stage III colorectal cancer, stage IV colorectal cancer, or not having colorectal cancer or polyps.

In embodiments, a number of different types of mathematical models can be employed. In embodiments, Model 1 uses the Universal Process Classification algorithm, a variant of K-Nearest Neighbors classification that utilizes a distance measurement to identify nearest neighbors. In embodiments, Model 2 uses support vector classifiers with radial basis function kernels during identification. In embodiments, Model 3 is a random forest classifier, an algorithm class that makes predictions by averaging the results of multiple randomly-initialized binary decision trees. In embodiments, Model 4 is a random forest classifier. In some embodiments, biometric data is included in the mathematical model. Biometric data includes height; Weight; BMI, Body Mass Index=(weight in kilograms/height in meters)/height in meters; Gender; Smoking status (nonsmoker, smoker, or ex-smoker); Alcohol consumption per week (0, 1-7, 8-14, 15-21, >21; and/or History of previous cancer (yes or no).

In some embodiments, the instructions when executed on a computing device comprise: receiving the detected amount of each of the polypeptides and/or nucleic acids coding for the polypeptides; retrieving a coefficient for each of the detected amounts of each of the polypeptides and/or nucleic acids coding for the polypeptides from a database; multiplying each of the detected amount by the corresponding coefficient to generate a weighted level for each of the polypeptides; analyzing the combination of weighted levels for each polypeptide with a model to determine the probability that the subject has colorectal cancer or is normal based on a change or lack thereof from the combination of predetermined weighted values of the polypeptides for normal subjects.

In some embodiments, access to a database of the profiles of the detected amount of each of the polypeptides and/or nucleic acids coding for the polypeptides for each of samples from subjects known to have low risk adenomatous polyps, high risk adenomatous polyps, stage I colorectal cancer, stage II colorectal cancer, stage III colorectal cancer, stage IV colorectal cancer, and from subjects known to not have colorectal cancer and/or stored mathematical models are available in a web based application. In embodiments, the profile of the detected amounts of the at least four polypeptides or nucleic acids coding for the polypeptides in the sample from the subject with unknown status is compared to the profile from subjects known to have low risk adenomatous polyps, high risk adenomatous polyps, stage I colorectal cancer, stage II colorectal cancer, stage III colorectal cancer, stage IV colorectal cancer, and from subjects known to not have colorectal cancer. In embodiments, the detected amounts of the at least four polypeptides or nucleic acids coding for the polypeptides in the sample from the subject with unknown status are analyzed with one or more mathematical models to generate a risk assessment.

In embodiments, each of the at least four reagents are attached to a solid surface. In embodiments, each of the reagents is attached to a different solid surface or a different location on a solid surface. In embodiments, the solid surface comprises a bead, a magnetic bead, and a slide, a well of a multiwell plate, a chip, a microfluidic channel or combinations thereof. In embodiments, each reagent is attached to a solid surface, each of the solid surfaces having a different internal marker. In embodiments, the different internal marker comprises a radioactive isotope tag, a quantum dot, a protein or peptide tag, an RFID tag, or a fluorescent dye. In embodiments each reagent is attached to a bead having a unique and different internal marker so that the presence or amount of each of polypeptides detected by the reagent is separately identifiable by the presence of the internal marker.

In embodiments, each of the at least four reagents is a primary antibody or antigen binding fragment specific for one of the at least four polypeptides that comprise GDF15, keratin 1-10, and two or more of hepsin, IL-8, CEA, L1CAM, MCP-1, and OPG. In some embodiments, the at least four polypeptides comprise at least 16 polypeptides comprising GDF15, keratin 1-10, CEA, L1CAM, hepsin, IL-8, AFP, CATD, CD44, ferritin, MIA, MDK, NSE, ON (SPARC), TWEAK, and YKL40. In other embodiments, a kit comprise at least four reagents that specifically bind to or detect at least four polypeptides or nucleic acids coding for the polypeptides. In other embodiments, each of the four reagents is a reagent that specifically detects a nucleic acid coding for one of the polypeptides. In embodiments, the reagents include a probe, a set or primers, a primer, or an aptamer.

In embodiments, the kit further comprises additional reagents for detecting additional polypeptides or nucleic acids coding for the polypeptides. In embodiments, each additional reagent specifically binds to one of the additional polypeptides or nucleic acids coding for the polypeptides that comprise MCP-1, OPG, AFP, ferritin, CATD, CD44, ALDH1A1, EPCAM, FAP, Galectin 3, IL-6, kallikrein 6, CEA, keratin 6, L1CAM, MIA, MDK, TWEAK, NSE, ON (SPARC), TGM2, VEGFA, or YKL40.

In embodiments, the kit further comprises at least four detectably labelled secondary reagents. Each detectably labelled secondary reagent specifically detects one of at least four polypeptides or nucleic acids coding for the polypeptides; and each of the at least four detectably labelled secondary reagents have a different detectable label. In some embodiments, a detectable label on a secondary reagent comprises a radioactive isotope, a fluorescent dye, an enzyme, a quantum dot, an enzyme, a luminescent reactant, or combinations thereof. In embodiments, the label on the detectably labelled secondary reagents differs from that of the internal marker on the solid surface.

In embodiments, each of the at least four detectably labelled secondary reagents is a secondary antibody or antigen binding fragment thereof. Each detectably labelled secondary antibody or antigen binding fragment thereof specifically binds to a one of the at least four polypeptides; and each of the at least four detectably labelled secondary antibodies or antigen binding fragments thereof has a different detectable label. In embodiments, each of the detectably secondary labelled antibody or antigen binding fragment binds to a different epitope than the primary antibody or antigen binding fragment thereof that binds to the same polypeptide.

In embodiments, a kit may include the at least four reagents, each in a separate container, at least two reagents of the at least four different reagents in a single container, or all of the at least four different reagents in a single container.

In embodiments, the kit comprises at least one standard comprising a known amount of at least one of the polypeptides and/or nucleic acid coding for the polypeptide. In some embodiments, at least four different standards are included in the kit, each standard having a known amount of one of the polypeptides and/or nucleic acid coding for the polypeptides. In embodiments, a standard for each of the polypeptides and/or nucleic acids coding for the polypeptides can be diluted to generate several samples having different known amounts of each of the polypeptides and/or nucleic acids coding for the polypeptides. In other embodiments, a known amount of all of the polypeptides being analyzed are in a single container. In embodiments, the standard can be lyophilized and instructions included in the kit for reconstitution and/or dilution.

In other embodiments, a standard comprises a low concentration quality control standard for each of the polypeptides or nucleic acids coding for the polypeptides. In other embodiments, a standard comprises a high concentration quality control standard for each of the polypeptides or nucleic acids coding for the polypeptides.

In embodiments, a standard can be a low concentration quality control sample, and/or a high concentration quality control standard. In some embodiments, the concentration of the polypeptide in the low quality control standard ranges from 0.001 to 500 ng/ml depending on the polypeptide. For example, a low concentration quality control standard for GDF15 is about 0.01 to about 0.5 ng/ml, a low concentration quality control standard for hepsin is about 2 to about 10 ng/ml, a low concentration quality control standard for IL-8 is about 0.1 to about 0.5 ng/ml, and a low concentration quality control standard for keratin 1-10 is about 100 to about 500 ng/ml.

In some embodiments, the concentration of the polypeptide in the high quality control standard ranges from 0.05 to 5000 ng/ml depending on the polypeptide. For example, a high concentration quality control standard for GDF15 is about 1 to about 5 ng/ml, a high concentration quality control standard for hepsin is about 20 to about 50 ng/ml, a high concentration quality control standard for IL-8 is about 0.1 to about 1 ng/ml, and a high concentration quality control standard for keratin 1-10 is about 1000 to about 5000 ng/ml.

In embodiments, the kit further comprises one or more control samples. In embodiments, the control samples comprise a sample from a subject known to have low risk polyps, a sample from a subject known to have high risk polyps, a sample from a subject known to have stage I colorectal cancer, a sample from a subject known to have stage II colorectal cancer, a sample from a subject having stage III colorectal cancer, a sample from a subject known to have stage IV colorectal cancer, a sample from a subject not known to have polyps or colorectal cancer, or combinations thereof. In embodiments, such control samples can be used to validate the method of detection of each of the polypeptides.

In embodiments, the standards and/or control samples are processed the same as the sample from the subject having unknown status.

In embodiments, once the at least four reagents and the detectably labelled secondary reagents are contacted with the samples, the amount of each of the polypeptides and/or nucleic acids coding for the polypeptides is detected by detecting the amount of the label on the secondary reagent. In embodiments, the label on the secondary reagent is detected using fluorescent activated cell sorting, absorbance at a specific wavelengths, detecting the amount of a radioactive isotope, and other methods of detecting the label. In some embodiments, when the at least four reagents are attached to a different solid surface, the at least four reagents can be separately analyzed from one another by detecting each internal marker for each of the four reagents. The amount of the detectably labelled secondary reagent for each of the at least four reagents can be determined to provide an amount of each of the at least four polypeptides and/or nucleic acids coding for the polypeptides using a standard curve based on the standard for the specific polypeptide.

A determination of the presence or detected amounts of each of the polypeptides and/or nucleic acids coding for the polypeptides is then analyzed using statistical methodology and/or mathematical modelling as described herein. The detected amount of each of the polypeptides and/or nucleic acids coding for the polypeptides can be increased or decreased as compared to a control from a subject not known to have polyps or colorectal cancer.

### EXAMPLES

### Example 1

A system allows for testing for the presence of multiple markers in serum in subjects that were classified as normal, having low risk adenomatous polyps, having high risk adenomatous polyps, having stage I, or having Stage II colorectal cancer. A number of different markers were tested and statistical analysis employed to identify combinations of markers that provided a high level of sensitivity to the risk of colorectal cancer.

### Materials

### Subject Samples

Colonoscopy-confirmed specimens were obtained from a hospital collection site. All samples were collected under approved protocols. The analyses in this study included 399 colonoscopy-confirmed subjects. The samples were stored at −80 ° C. and were thawed immediately prior to testing and diluted into proper working range for each analyte assayed. Bead assay kits and reagents

All dilution buffers and read buffers were from Millipore Sigma® or Ray Biotech®. Antibodies specific for each marker were also obtained from Millipore Sigma® or Ray Biotech®. Capture antibodies were attached to magnetic beads such as XMAP® magnetic beads. The magnetic beads contain sets of internally coded different fluorescent beads. Reporter molecules with different fluorescent tags were obtained from Millipore Sigma®. The assay reader is MAGPIX™ by LUMINEX®.

Multiplexed calibrator sets were prepared for each panel. Each standard curve consisted of 8 points spanning the full range of the assay, including an assay blank. Standards were prepared with antibodies spiked into the appropriate sample diluent containing the equivalent serum concentration that is present in diluted samples to reflect the diluent/serum composition in the diluted patient samples. Prediluted standards were stored at −80 ° C.

### Methods

### Ligand Binding Assay

Assays for 1-9 different biomarkers were conducted using the Luminex® MAGPIX multiplex instrument. Custom assay kits were prepared by Millipore Sigma® to include cancer-related markers selected for this study. The markers evaluated included: GDF 15, DKK1, NSE, ON (SPARC), Periostin, TRAPS, OPG, YKL40, TWEAK, AFP, Leptin, TNFa, OPN, VEGF, Cortisol, Keratin 6, Keratin 1-10, IL-6, IL-8, MCP-1, L1CAM, Mesothelin, MDK, Hepsin, Kallikrein 6, TGM2, ALDH1A1, EpCAM, CD44, TIM3, Galectin-3, CATD, FAP (Seprase), MIA, MPO, SHBG, Ferritin, and ACT. Antibodies specific for each biomarker were attached to a specific set of colored coded magnetic beads by Millipore®. Diluted subject samples, working standards, Quality control samples, and the magnetic bead master mix were diluted according to manufacturer's instruction. The subject sample, standards, control samples and beads were added to a 96-well plates (Millipore Sigma®) and incubated overnight at 4° C.

Following the incubation, the plates were warmed to room temperature and then washed with assay buffer. Once the plates are washed, 25 uL of prediluted blends of detection antibodies are added to each well for 60 mins with continuous shaking at 650 rpm, plates were washed 3 times in wash buffer, and Read Buffer was loaded in each well. Plates were immediately read on Luminex® MAGPIX multiplex instrument. Statistical analysis

The data were analyzed by graphing the best-fit standard curve and matching the serum sample values on the curve. Milliplex® Analyst software was used to calculate CV % of duplicate assays, and to apply the correct dilution factor for any serum samples diluted. The data is further analyzed using models that were designed to have a sensitivity to early stage cancer of at least 90% or greater; a specificity for healthy normal of at least 50% or greater; and an Area under the Curve (AUC) of at least 80% or greater. Several different models were analyzed using different combinations of markers. Results

The amount of each of the markers found in each of 399 samples was reported as the median picograms or nanograms per ml for normal samples, low risk adenomatous polyps, high risk adenomatous polyps, stage I samples, and stage II sample. The results are shown in Table 1.

TABLE 1

| | | | Feb. 6, 2018 Reported Values | | |
|---|---|---|---|---|---|
| | Apr. 7, 2018 Reported Values | | Diagnosis | | |
| | Adenoma risk | | | STAGE | STAGE |
| Biomarker | LOW | HIGH | NORMAL | I | II |
| AFP (pg/mL) | 2425.74 | 3189.52 | 11642.57 | 2957.90 | 2144.78 |
| Leptin (pg/mL) | 35459.93 | 23196.10 | 18649.05 | 15397.98 | 13501.89 |
| TNFa (pg/mL) | 28.70 | 23.61 | 10.00 | 9.71 | 10.75 |
| OPN (pg/mL) | 16233.41 | 16436.61 | 16993.48 | 16273.38 | 18347.98 |
| VEGF (pg/mL) | 188.03 | 193.87 | 107.46 | 107.22 | 107.43 |
| Cortisol (ng/mL) | 125.08 | 83.51 | 63.40 | 61.51 | 54.80 |
| Keratin 6 (ng/mL) | 25.56 | 52.92 | 70.37 | 56.01 | 40.40 |
| Keratin 1-10 (ng/mL) | 77.31 | 139.97 | 3852.00 | 530.24 | 345.05 |
| IL-6 (pg/mL) | 14.93 | 12.01 | 3.29 | 3.59 | 4.61 |
| IL-8 (pg/mL) | 13.04 | 14.86 | 15.63 | 11.58 | 16.61 |
| MCP-1 (pg/mL) | 575.59 | 718.49 | 732.35 | 787.02 | 903.99 |
| L1CAM (ng/mL) | 5.67 | 5.93 | 6.83 | 5.41 | 6.09 |
| Mesothelin (ng/mL) | 22.19 | 20.22 | 19.57 | 20.78 | 19.89 |
| Midkine (pg/mL) | 488.31 | 408.02 | 331.67 | 296.33 | 314.46 |
| Hepsin (ng/mL) | 1.22 | 1.50 | 1.44 | 0.90 | 1.00 |
| Kallikrein 6 (pg/mL) | 5172.67 | 5214.61 | 5540.75 | 5320.21 | 5199.79 |
| TGM2 (ng/mL) | 8.00 | 5.32 | 4.40 | 3.34 | 3.79 |
| ALDH1A1 (ng/mL) | 138.41 | 103.56 | 232.65 | 243.33 | 212.17 |
| EpCAM (pg/mL) | 1172.31 | 900.92 | 893.55 | 765.19 | 815.35 |
| CD44 (ng/mL) | 13.38 | 13.01 | 11.69 | 10.88 | 11.76 |
| TIM3 (pg/mL) | 3412.22 | 3595.64 | 4623.76 | 4929.25 | 4876.26 |
| GDF15 (ng/mL) | 0.72 | 0.81 | 0.79 | 0.87 | 0.91 |
| DKK1 (ng/mL) | 2.67 | 2.35 | 2.26 | 2.72 | 2.42 |
| NSE (ng/mL) | 5.72 | 5.11 | 4.18 | 4.44 | 4.42 |
| ON (SPARC) (ng/mL) | 1113.36 | 1087.21 | 756.06 | 761.94 | 706.02 |
| Periostin (ng/mL) | 78.64 | 79.03 | 67.32 | 69.85 | 64.79 |
| TRAP5 (ng/mL) | 32.97 | 30.75 | 26.46 | 27.26 | 25.88 |
| OPG (ng/mL) | 0.70 | 0.76 | 0.65 | 0.74 | 0.70 |
| YKL40 (ng/mL) | 91.92 | 83.52 | 64.08 | 86.12 | 90.32 |
| TWEAK (ng/mL) | 0.84 | 0.88 | 0.91 | 0.90 | 0.91 |
| Galectin-3 (ng/mL) | 6.54 | 6.75 | 4.24 | 4.64 | 4.20 |
| Cath D (ng/mL) | 130.52 | 110.79 | 79.16 | 88.18 | 96.47 |
| FAP (Seprase) (ng/mL) | 113.50 | 107.93 | 105.93 | 106.40 | 97.08 |
| MIA (ng/mL) | 18.05 | 17.80 | 16.24 | 16.73 | 14.41 |
| MPO (ng/mL) | 106.62 | 72.82 | 74.41 | 75.18 | 75.92 |
| SHBG (nM) | 73.11 | 73.64 | 69.08 | 68.80 | 77.22 |
| Ferritin (ng/mL) | 681.29 | 557.70 | 422.30 | 475.56 | 291.69 |
| ACT (pg/mL) | 14336.41 | 13296.35 | 19390.16 | 20578.42 | 20395.11 |

The markers showing the largest differences between normal and Stage I or Stage II colorectal cancer, regardless of whether the marker amount was increased or decreased, include: AFP, leptin, ferritin, anti-chymotrypsin (ACT), TIM3, OPN, Kallikrein 6, EPCAM, and MCP-1.

This data was further analyzed using three different mathematical models and using different combination of markers as well as biometric data (in model 1). Model 1 uses the Universal Process Classification algorithm, a variant of K-Nearest Neighbors classification that utilizes a distance measurement to identify nearest neighbors. Model 2 uses well-established support vector classifiers with radial basis function kernels during identification. Model 3 is a random forest classifier, an algorithm class that makes predictions by averaging the results of multiple randomly-initialized binary decision trees. Model 4 is also a random forest classifier In model 1, biometric data used included height (note, either height and weight or BMI were used, not both);Weight (note, either height and weight or BMI were used, not both); BMI, Body Mass Index =(weight in kilograms/height in meters)/height in meters; Gender; Smoking status (nonsmoker, smoker, or ex-smoker); Alcohol consumption per week (0, 1-7, 8-14, 15-21, >21; and History of previous cancer (yes or no).The results are shown in Table 2.

Model four is also a random forest classifier. It uses the following 15 markers: AFP, CATD, CD44, Ferritin, GDF15, Hepsin, IL-8, Keratin 1-10, L1CAM, MIA, MDK, NSE, ON (SPARC), TWEAK, YKL40.

TABLE 2

| Model 1 | Model 2 | Model 3 | Model 4 |
|---|---|---|---|
| ACT | ACT | ACT | |
| AFP | AFP | AFP | AFP |
| | | ALDH1A1 | |
| Cath D | Cath D | Cath D | Cath D |
| CD44 | CD44 | CD44 | CD44 |
| DKK1 | DKK1 | DKK1 | |
| EpCAM | | | |
| FAP | | | |
| Ferritin | Ferritin | Ferritin | Ferritin |
| GALECTIN-3 | | | |
| GDF15 | GDF15 | GDF15 | GDF15 |
| Hepsin | Hepsin | Hepsin | Hepsin |
| | IL-6 | | |
| IL-8 | IL-8 | IL-8 | IL-8 |
| | Kallikrein 6 | | |
| | Keratin 1-10 | Keratin 1-10 | Keratin 1-10 |
| Keratin 6 | Keratin 6 | | |
| | L1CAM | | L1CAM |
| MCP-1 | MCP-1 | MCP-1 | |
| | MIA | | MIA |
| | MDK | | MDK |
| MPO | MPO | MPO | |
| | NSE | | NSE |
| | ON (SPARC) | | ON (SPARC) |
| OPG | OPG | OPG | |
| | TGM2 | | |
| TIM3 | TIM3 | | |
| | TWEAK | | TWEAK |
| | VEGF-A | | |
| | YKL40 | | YKL40 |

Model 1 provided a 99% sensitivity, a 56% specificity, and 83% AUC. Model 2 provided 97% sensitivity, 23% specificity, and 74% AUC. Model 3 provided 99% sensitivity, 50% specificity, and 80% AUC. Model 4 provided 91% Sensitivity, 48% Specificity, and 82% AUC, with an Fl score of 0.88

All four of the models included some of the same markers including: AFP, CathD, CD44, ferritin, GDF15, hepsin, and 11-8. Models 1, 2, and 3 also included ACT, DKK1, MCP-1, MPO, and OPG. Models 2, 3, and 4 also included keratin 1-10. Models 1 and 2 further included keratin 6 and TIM3. Models 2 and 4 further included L1CAM, MIA, MDK, NSE, ON (SPARC), TWEAK, and YKL40. Model 1 also included markers EpCAM, FAP, and Galectin 3. Model 1 included biometric parameters. Model 2 also included markers IL-6, kallikrein 6, and VEGF-A. Model 3 further included marker ALDH1A1.

### CONCLUSIONS

A marker panel was identified that was useful to distinguish normal serum samples from subjects having low risk adenomatous polyps, high risk adenomatous polyps, Stage I or Stage II cancers using four different models. Two of the models provided a sensitivity of at least 90%, a specificity of at least 50%, and an AUC of at least 80%. A set of 12 markers was shared between all 3 models. These markers as well as others can be utilized in a test to screen subjects for the risk of having colorectal cancer.

What is claimed:

1. A kit for detecting one or more markers in a subject of an unknown status comprising:
at least two reagents, each of the two reagents specifically binds to one of a plurality of polypeptides in a sample from the subject, the plurality of polypeptides comprising IL-8 and ferritin; and
at least one standard comprising a known amount of one of the polypeptides.

2. The kit of claim 1, further comprising one or more non-transitory computer-readable media having computer-executable instructions embodied thereon that, when executed by one or more computing devices, cause the computing devices to:
receive the detected amount of each of the polypeptides;
retrieve a coefficient for each of the detected amounts of each of the polypeptides from a database;
multiply each of the detected amounts of the polypeptides by the corresponding coefficient to generate a weighted level for each of the polypeptides;
analyze the combination of weighted levels for each polypeptide with a machine learning model to determine the probability that the subject has colorectal cancer based on a change or lack thereof from a combination of predetermined weighted values of the polypeptides for normal subjects.

3. The kit of claim 1, further comprising at least two detectably labelled secondary reagents, each detectably labelled secondary reagent specifically binds to one of the polypeptides, and each of the at least two detectably labelled secondary reagents has a different detectable label.

4. The kit of claim 3, wherein the detectable label comprises a radioactive isotope, a fluorescent dye, an enzyme, a quantum dot, a luminescent reactant, or combinations thereof.

5. The kit of claim 1, wherein the plurality of polypeptides comprises IL-8, ferritin, GDF15, keratin 1-10, and hepsin.

6. The kit of claim 1, wherein the plurality of polypeptides comprises IL-8, ferritin, GDF15, keratin 1-10, CEA, L1CAM, MCP-1, and OPG.

7. The kit of claim 1, wherein the plurality of polypeptides comprises IL-8, ferritin, GDF15, keratin 1-10, CEA, L1CAM, MCP-1, OPG, and hepsin.

8. The kit of claim 1, wherein the plurality of polypeptides further comprises one or more of AFP, CATD, CD44, MIA, MDK, NSE, ON (SPARC), TWEAK, and YKL40.

9. The kit of claim 8, wherein the plurality of polypeptides further comprises MCP-1 and OPG.

10. The kit of claim 1, wherein the plurality of polypeptides further comprises CEA.

11. The kit of claim 1, wherein the at least two reagents comprise at least two primary antibodies or antigen binding fragments thereof, each primary antibody or antigen binding fragment thereof specifically binds to one of a plurality of polypeptides.

12. The kit of claim 3, wherein the at least two detectably labelled secondary reagents comprise at least two secondary antibodies or antigen binding fragments thereof; each detectably labelled secondary antibody or antigen binding fragment thereof specifically binds to one of the plurality of polypeptides at a different epitope than the primary antibody or antigen binding fragments; and each of the at least two detectably labelled antibodies or antigen binding fragments thereof has a different detectable label.

13. The kit of claim 1, wherein each of the at least two reagents is attached to a solid surface, wherein the solid surface comprises a bead, a magnetic bead, a well, slide, a tube, or combinations thereof.

14. The kit of claim 13, wherein each of the at least two reagents is attached to a different solid surface comprising a magnetic bead with a different internal marker.

15. The kit of claim 14, wherein the different internal marker comprises a fluorescent dye, a quantum dot, a protein tag, a RFID tag, or combinations thereof, and wherein the internal marker of the solid surface is different from the detectable label of the detectably labelled secondary reagent specific for polypeptide or nucleic acid coding for the polypeptide attached to the solid surface.
